(19) United States
(12) Patent Application Publication
(10) Pub. No.: US 2022/0395618 A1
(43) Pub. Date: Dec. 15, 2022

(54) Signal Activated Method for Draining an Effluent Bag and Devices

(71) Applicant: Fresenius Medical Care Deutschland GmbH, Bad Homburg (DE)

(72) Inventor: Juergen Klewinghaus, Oberursel (DE)

(21) Appl. No.: 17/775,687

(22) PCT Filed: Nov. 10, 2020
(86) PCT No.: PCT/EP2020/081603

§ 371 Date: May 10, 2022

(30) Foreign Application Priority Data
Nov. 12, 2019 (DE) 10 2019 130 434.2

(51) Int. Cl. A61M 1/34; A61M 1/36; A61M 1/14; A61M 1/16

(52) U.S. Cl. CPC A61M 1/341; A61M 1/3448; A61M 1/3672; A61M 1/155; A61M 1/1566; A61M 1/1635; A61M 2205/3334; A61M 2205/18

(57) ABSTRACT

The present disclosure relates to a control device or closed-loop control device, programmed to control or regulate a blood treatment apparatus during a treatment of a patient's blood carried out in a treatment session using an extracorporeal blood tubing set and the blood treatment apparatus while balancing liquid flows and conveying via different liquid pumps. The control device or closed-loop control device is further programmed to interrupt the balancing and/or the liquid flows at one or more predetermined interruption time points which lie within the duration of the treatment session.

### CROSS-REFERENCE TO RELATED APPLICATIONS

The present application is the national stage entry of International Patent Application No. PCT/EP2020/081603, filed on Nov. 10, 2020, and claims priority to Application No. DE 10 2019 130 434.2, filed in the Federal Republic of Germany on Nov. 12, 2019, the disclosures of which are expressly incorporated herein in its entirety by reference thereto.

### FIELD OF THE DISCLOSURE

The present disclosure relates to a control device or closed-loop control device according to the present disclosure, a blood treatment apparatus according to the present disclosure and an effluent bag draining apparatus according to the present disclosure. Further, it relates to a system according to the present disclosure. It also relates to a digital storage medium according to the present disclosure, a computer program product according to the present disclosure, a computer program according to the present disclosure, furthermore, a method for the draining of an effluent bag according to the present disclosure, or according to generic terms related to these embodiments.

### BACKGROUND

Extracorporeal blood treatment is known in the prior art. Whereby the patient's blood is taken and fed along an extracorporeal blood circuit and through, for example, a blood filter. The blood filter includes a blood chamber through which blood is guided, and a dialysis liquid chamber, through which dialysis liquid is guided. Both chambers are separated from each other by a semi-permeable membrane. Blood and dialysis liquid are mostly guided through the blood filter by the counter current principle. The blood is purified in the blood filter, on exiting the blood filter the dialysis liquid, from now on referred to as dialysate, is regarded as used and is discarded. In addition to the dialysate, the fluid to be discarded also encompasses filtrate, which encompasses water that has been withdrawn from the blood in the blood filter. Filtrate and dialysate will be referred to individually or collectively in the following simply as effluent.

The effluent is, in the prior art, directly discarded via an effluent outlet line or, especially during an acute treatment, fed to an effluent bag and is initially stored therein. After completion of the blood treatment, or in bag draining intervals during the blood treatment (intervals in which the bag is emptied), the effluent is discarded from the effluent bag for example into a washbasin or into any other type of sink.

### SUMMARY

An object of the present disclosure can be seen as the specification of a control device or closed-loop control device, a blood treatment apparatus and an effluent bag draining apparatus, furthermore a system made with consisting of or having both of the latter apparatuses.

Furthermore, a digital storage medium, a computer program product as well as a computer program should be specified.

Further, a method for the draining of the effluent bag should be specified which is practicable with the system referred to above.

The object according to the present disclosure is achieved by an control device or closed-loop control device having the features of the present disclosure, a blood treatment apparatus having the features of the present disclosure and an effluent bag draining apparatus having the features of the present disclosure. Furthermore, it is achieved by a system having the features of the present disclosure. It is further achieved by a digital storage medium having the features of the present disclosure, a computer program product having the features of the present disclosure, a computer program having the features of the present disclosure, as well as by a method for the draining of an effluent bag having the features of the present disclosure.

The present disclosure thus relates to a control device or closed-loop control device which is programmed for controlling or regulating a blood treatment apparatus for treating the blood of a patient. The treatment is performed within a treatment session, which is optionally performed using an extracorporeal blood tubing set and by filling an effluent bag with effluent. During the treatment session, liquid flows (e.g., the flows of calcium solution, citrate solution, substituate solution (in pre-dilution and/or in post-dilution), Heparin solution, net ultrafiltration rate, dialysis flow rate, in each case when applicable or given), which are generated by a calcium pump, a citrate pump, a substituate pump, a Heparin pump, a filtrate pump and/or by a dialysis liquid pump of the blood treatment apparatus are preferably balanced.

During the blood treatment session and/or within the duration of the blood treatment session, the control device or closed-loop control device according to the present disclosure is further programmed to be able to temporarily interrupt and restart the balancing, the flow via one or more of the pumps used, in any combination, e.g., the calcium solution flow (or the conveying action the calcium pump), the citrate solution flow (or the conveying action of the citrate pump), the Heparin solution flow (or the conveying action of the Heparin pump), the net ultrafiltration flow (or the conveying action of the filtration pump), the substituate flow (or the conveying action of the substituate pump) and/or the dialysis liquid flow (or the conveying action of the dialysis liquid pump).

The control device or closed-loop control device is programmed to prompt such an interruption, preferably automatically, that is without human intervention, at one or more predetermined interruption time points during the treatment session, i.e. within the duration of the treatment.

The blood treatment apparatus according to the present disclosure encompasses such a control device or closed-loop control device according to the present disclosure.

The effluent bag draining apparatus according to the present disclosure encompasses an effluent outlet line, which can be connected in fluid communication to an effluent bag and which may herein be referred to also as a drain line for leading effluent out of an effluent bag. The effluent bag draining apparatus also encompasses a pump section to actively convey effluent along an effluent outlet line and thereby out of the effluent bag.

A control device (which is optionally configured to also regulate) is also encompassed by the effluent bag draining apparatus according to the present disclosure. The control device is programmed to prompt the pump section to convey effluent out of the effluent bag when (or as soon as) a predetermined draining time point is reached.

The system according to the present disclosure encompasses a blood treatment apparatus according to the present disclosure and an effluent bag draining apparatus according to the present disclosure.

The digital storage medium according to the present disclosure is configured so as to interact with a programmable computer system in such a way that a control device or closed-loop control device of a blood treatment apparatus is reprogrammed into a control device or closed-loop control device according to the present disclosure, whereby the blood treatment apparatus becomes a blood treatment apparatus according to the present disclosure, and/or that a control device of an effluent bag draining apparatus is programmed in such a way that the effluent bag draining apparatus becomes an effluent bag draining apparatus according to the present disclosure.

A digital storage medium according to the present disclosure, may be provided such as in the form of a diskette, CD or DVD, EPROM, FRAM or SSD with electronically readable control signs.

The computer program product according to the present disclosure may be embodied as a signal wave or with a program code stored on a machine readable carrier in order to interact with a programmable computer system so that a control device or closed-loop control device is reprogrammed into a control device or closed-loop control device according to the present disclosure and/or that a control device is reprogrammed into a control device of an effluent bag draining apparatus according to the present disclosure. The computer program according to the present disclosure encompasses a program code that prompts or results in the device or closed-loop control device being reprogrammed into a control device or closed-loop control device according to the present disclosure and/or that a control device be reprogrammed into a control device of an effluent bag draining apparatus according to the present disclosure, when the computer program is running on a computer.

The method according to the present disclosure for draining an effluent bag, which is filled with effluent during the patient's blood treatment, takes place on a blood treatment apparatus according to the present disclosure and an effluent bag draining apparatus according to the present disclosure or on a system according to the present disclosure, whereby an effluent bag which includes an effluent outlet opening is provided on the blood treatment apparatus or on the effluent bag draining apparatus.

Thereby, the effluent outlet opening of the effluent bag is connected to the effluent outlet line of the effluent bag draining apparatus in order to lead effluent out of the effluent bag immediately or later. Should a shut-off element for the effluent outlet opening be provided, then by opening the shut-off element a fluid communication between the interior of the effluent bag and the interior of the effluent outlet line may be established.

The method encompasses stopping or interrupting the balancing, the ultrafiltrate flow (or the conveying action) of at least one filtrate pump, the substituate flow (or the conveying action) of the substituate pump and/or the dialysis liquid flow (or the conveying action) of the dialysis liquid pump or one of the other pumps mentioned herein at (or starting at) one or more predetermined interruption time points which lie within the treatment session, i.e. within the duration of the treatment.

The method further encompasses prompting the pump section to convey effluent out of the effluent bag when (or as soon as) a predetermined and/or defined draining time point is reached. The initiation of these two steps (stopping when an interruption time point is reached or initiation of conveying when an draining time point is reached) is optionally automatic, e.g., prompted by or triggered by the control device or closed-loop control device of the treatment apparatus and/or by the control device of the effluent bag draining apparatus.

Embodiments according to the present disclosure may include some, any or all of the following features in any combination, unless the person skilled in the art recognizes their combination as technically impossible.

In all of the statements made previously and in the following, the use of the expression “may be” or “may have” and so on, is to be understood synonymously with “preferably is” or “preferably has,” and so on respectively, and is intended to illustrate embodiments according to the present disclosure.

Whenever numerical words are mentioned herein, the person skilled in the art shall recognize or understand them as indications of numerical lower limits. Unless it leads the person skilled in the art to an evident contradiction, the person skilled in the art shall comprehend the specification for example of “one” as encompassing “at least one”. This understanding is also equally encompassed by embodiments of the present disclosure as the interpretation that a numeric word, for example, “one” may alternatively mean “exactly one”, wherever this is evidently technically possible for the person skilled in the art. Both are encompassed by embodiments of the present disclosure and apply herein to all used numerical words.

Whenever an embodiment is mentioned herein, it is then an exemplary embodiment according to the present disclosure.

When it is disclosed herein that the subject-matter according to the present disclosure includes one or several features in a certain embodiment, it is also respectively disclosed herein that the subject-matter according to the present disclosure does, in other embodiments, likewise according to the present disclosure, explicitly not include this or these features, for example, in the sense of a disclaimer. Therefore, for every embodiment mentioned herein it applies that the converse embodiment, e.g., formulated as negation, is also disclosed.

In several embodiments, the interruption is made by the control device or closed-loop control device according to the present disclosure, or prompted by the device itself, each for a predetermined interruption time period (or: interruption interval), which each begins when the interruption time point that initiates it is reached. Successive interruption time periods are preferably of constant length, but may alternatively be of different lengths.

In some embodiments wherein upon reaching the predetermined interruption time point a notification or alarm may be given to the user prompting him to manually empty the effluent bag, for example a notification on the display and/or an optical and/or an audible alarm (light source, lamp, loudspeaker, etc.)

During the blood treatment session, the effluent bag is in fluid communication with the blood treatment apparatus and accumulates effluent produced during the treatment session. Thereto, it may optionally be in fluid communication with the blood treatment apparatus and/or with the effluent bag draining apparatus. For this purpose, it may be held, carried or received by the blood treatment apparatus and/or by the effluent bag draining apparatus.

In several embodiments, the control device or closed-loop control device according to the present disclosure is in signal communication with a signal emitter or with a clock e.g., an internal clock (i.e. a clock of the control device or closed-loop control device). The clock may alternatively be an external clock, such as a clock of the blood treatment apparatus, a network clock, a ward clock or an atomic clock.

The control device or closed-loop control device is hereby configured, upon receiving a predetermined signal from the signal emitter or upon reaching a predetermined time on the clock, to specify or establish the time the signal is received or the predetermined time is reached as an interruption time point (beginning of the interruption interval).

The control device or closed-loop control device, along with the control device of the effluent bag draining apparatus described below, may be programmed, in response to a signal from the signal emitter, or at a predetermined time point (e.g., measured with one of the clocks mentioned herein) to immediately start an interruption time period (or interval) or in the case of the effluent bag draining apparatus, a draining time period (or interval) or to start it after a predetermined period, or rest or waiting period has elapsed.

In some embodiments, the control device or closed-loop control device according to the present disclosure is programmed to control or regulate the blood treatment apparatus in such a way as to ensure that the effluent bag is not filled above a predetermined level or volume, especially, that it does not burst and cause any contamination.

For example, the fill volume of the effluent bag can be monitored using weighing scales or the fill level can be monitored using a sensor. If, for example, a predetermined weight or a predetermined level is reached or exceeded, the control device or closed-loop control device, can prompt, such as automatically, the balancing and/or at least one of the aforementioned pumps to be stopped and the effluent bag, for example, to be automatically drained via the effluent bag draining apparatus. The control device or closed-loop control device can further be programmed to prompt a subsequent continuation of the blood treatment session, the balancing, the pump activity etc., for example also automatically.

Hereby, the control device or closed-loop control device should optionally control and/or optionally regulate in such a way that the effluent bag is not filled above a predetermined level, e.g., if a draining interval was skipped, which can also be manually prompted. In these embodiments the detected level in the effluent bag and/or the detected weight of the effluent bag can be used to begin a draining. Appropriate monitoring of level and/or weight by the blood treatment apparatus may be provided. If an operating condition should occur in which the draining using timings (i.e. taking into consideration the interruption times) is not sufficient and for example the weighing scales or the level sensor detect that the effluent bag is full or too full, then an interruption interval can be initiated or triggered automatically. In this case, for example, the user can be prompted to manually start the draining process.

Monitoring, controlling or regulating can provide, for example, for determining and/or monitoring an effluent bag fill level or weight, using data from pumps, weighing scales, or sensors in signal communication with the blood treatment apparatus or the effluent bag draining apparatus. Monitoring may include a comparing with target values and/or maximum values.

In several embodiments the blood treatment apparatus is configured as a hemodialysis apparatus, hemofiltration apparatus or hemodiafiltration apparatus, such as an apparatus for the acute, chronic renal replacement therapy or for the continuous renal replacement therapy (CRRT).

In some embodiments, the control device of the effluent bag draining apparatus according to the present disclosure is programmed to convey effluent out from the effluent bag using the pump section over a predetermined draining time period out through (or via) the effluent outlet line.

In several embodiments, the control device effluent bag draining apparatus is in signal communication with a signal emitter or with a clock. The clock can, e.g., be a clock of the effluent bag draining apparatus, it may also be on the effluent bag draining apparatus. The clock can be an external clock, e.g., the optional clock of the blood treatment apparatus, a network clock, a ward clock, or an atomic clock. The control device is hereby configured, upon receiving a predetermined signal from the signal emitter or upon reaching a predetermined time on the clock (or possibly, after receiving a corresponding signal from the clock) to specify the time point at which the signal is received or the predetermined time is reached as an interruption time point or to set it in relation to this, e.g., as a function of time.

In some embodiments of the system according to the present disclosure the blood treatment apparatus and the effluent bag draining apparatus are provided separately from one another, such as during the patient's treatment which is carried out by the blood treatment apparatus and/or during the draining of the effluent bag, apart from a fluid communication between effluent bag and blood treatment apparatus and/or between effluent bag and effluent bag draining apparatus.

In several embodiments of the system the control device or closed-loop control device of the blood treatment apparatus and the control device of the effluent bag draining apparatus are each programmed in such a way that at least one interruption time point and at least one draining time point occur simultaneously or offset by a predetermined period of time or waiting period have a predetermined temporal relationship to each other.

The system may be configured such that the interruption intervals of the blood treatment apparatus correspond to the draining intervals of the effluent bag draining apparatus, e.g., have a temporal relationship, in order to trigger the draining of an effluent bag of the effluent bag draining apparatus, i.e. a draining interval, precisely when there is an interruption interval on the blood treatment apparatus.

In some embodiments of the system the control device or closed-loop control device of the blood treatment apparatus and the control device of the effluent bag draining apparatus are each programmed in such a way that at least one interruption time period (or an interruption interval) and at least one draining time period (or a draining interval) overlap each other, such as by at least 50%, 60%, 70%, 80% or 90% of the duration of either of them.

In some embodiments of the system, the clock which is in signal communication with the control device or closed-loop control device of the blood treatment apparatus, and the clock which is in signal communication with the control device of the effluent bag draining apparatus, are identical or are synchronised, i.e. it is either one and the same clock or two clocks which display, indicate or give identical times or at least comparable times. Alternatively, the system can know how large the generally constant deviation of the two times is from one another. For example, the two clocks are based on the same time zone (e.g., Central European Time (CET)).

In several embodiments, the predetermined interruption time point and/or the predetermined draining time point each occurs at a predetermined time, for example, every hour (e.g., 3:00 p.m., 4:00 p.m., etc.), every half hour (e.g., 3:00 p.m., 3:30 p.m., 4:00 p.m., 4:30 p.m., etc.), etc.

In some embodiments, the predetermined interruption time point or the predetermined draining time point is reached a predetermined number of units of time after a defined event (e.g., minutes, half hours, quarter hours or parts thereof). So for example, any time point may be fixedly or changeably predefined by the manufacturer or the user and may be, for example 30 minutes, e.g., after the start of the treatment session; after pressing the start button; after the first starting or stopping of the balancing, the ultrafiltration pump or another pump or after a first, second, third interruption or drain time point preceding this interruption time point or draining time point, etc.

In several embodiments an interruption time duration (or an interruption interval) begins with an interruption time point and/or a draining time period (or draining interval) begins with a draining time point.

In several embodiments, the pump section of the effluent bag draining apparatus according to the present disclosure, is a disposable or a single-use item.

In some embodiments the pump section includes an impeller or a pump rotor.

In several embodiments the effluent outlet line is in conveying connection with the pump section.

In several embodiments, the pump section is connected to or can be connected to a pump drive that can optionally be detached therefrom.

In some embodiments, the pump section or the pump drive includes a rechargeable power source, e.g., a rechargeable battery.

In some embodiments of the method according to the present disclosure, the effluent is conveyed out of the effluent bag using the pump section.

In several embodiments of the method, the opening of the shut-off element is carried out manually, in others it is carried out automatically.

In some embodiments, the effluent bag can be a container of any type, for example, a container with a flexible outer skin such as a film (or plastic film), or can be made of film, have a hard outer skin such as a canister, etc.

Suitable, optional tube connectors, or pairs of connectors, can be provided in order to facilitate the connection of the effluent outlet line to the effluent bag and/or to the sink.

In several embodiments, the pump section is magnetically mounted or driven and includes the pump head. This pump head is configured, for example, as an impeller pump head or as its rotor. This electrically insulated type of mounting or driving serves to protect the patient from an electric shock.

In some embodiments, the pump drive is manually connected to the pump section that includes the pump head.

In several embodiments, the pump drive is optionally configured with a magnetic section, e.g., configured as coils with an iron core. This serves for the magnetic coupling and/or magnetic driving of components, such as a magnetically driven pump rotor. The drive is preferably contactless.

Some embodiments encompass an induction charging station for charging the rechargeable battery of the pump drive. The induction charging station optionally has control electronics which are configured to detect when the pump drive is resting on the induction charging station and to charge the rechargeable battery.

The induction charging station can be or include a platform or a base for the pump drive.

The present disclosure relates to an effluent bag draining apparatus which includes at least one pump section with a pump rotor. The pump section is provided to be functionally connected or combined with a pump drive. The pump section, and optionally also all other sections of the effluent bag draining apparatus, do not, in some embodiments include a device (devices) for connecting the pump section to the pump drive, except for an optional magnetic connection and/or the use of gravity.

In several embodiments, the pump drive includes an induction charging coil for charging the power source or rechargeable battery.

In some embodiments, the pump drive housing includes a pump drive installing section for installing the pump drive on a pump drive installation surface.

In several embodiments, the pump drive housing includes a connecting section for, such as functionally, connecting the pump drive to the pump section, which contains a pump rotor. Hereby, the connecting section can be a cavity, one or more openings, one or more blind openings or the like.

In some embodiments, the pump drive installation section and the connecting section are located at, or allocated to, opposite ends of the pump drive housing.

In several embodiments, the pump drive has at least one, such as a coloured, lighting device. For example, this can be embodied as a ring, such as an LED or LED-coloured ring.

The lighting device can, for example, be positioned in or on the pump drive housing.

In some embodiments, the control electronics of the pump drive are embodied with a wireless module or connected to such a one.

In several embodiments, the control electronics are embodied or connected with at least one motion sensor.

In several embodiments, the control electronics are configured to switch off the lighting device when or after the voltage source or battery has been fully recharged.

In several embodiments, the preferred electrical power rating is between 25 and 40 watts (W), such as 30 to 35 W, preferably 32 W.

In some embodiments, the operating voltage of the electrical drive of the pump is 24 V.

In several embodiments, the pump section has a device for releasably attaching it to a section of the effluent bag draining apparatus, such as for releasably attaching it to a installation section of the effluent bag draining apparatus or at the base of the blood treatment device

In some embodiments, the device for releasably attaching the pump section is a clamping device or a snap-in or clip device or includes such a device.

In some embodiments, the device for releasably attaching the pump section, which is optionally a clamping device or a snap-in or clip device has a curved form.

The curved form can take the shape of a half-channel curved along its longitudinal extension.

In some embodiments, the pump drive, pump section and/or another part of the pump does not in any instance include any mechanical connection device to connect the pump drive to the pump section, except for the pump section's installation surface on which the pump drive is positioned in order to achieve the functioning pump.

One advantage that can be achieved in this way may be that the pump drive can, at any time, simply be pulled up or lifted off (even while the pump is running). This can be done with one hand, especially if no mechanical connecting devices have to be opened beforehand, as the latter are not provided in these embodiments.

In several embodiments, the pump section is connected with its liquid inlet or fluid inlet to a tube section leading from the effluent bag to the pump.

In some embodiments, the pump section is connected with its liquid outlet or fluid outlet to the drain line, which can lead to a drain, basin or sink for discarding the effluent

In several embodiments, the pump rotor is magnetically mounted and/or magnetically driven.

The magnetic section of the pump rotor may be a permanent magnet.

In several embodiments, the storage capacity of the rechargeable battery of the pump drive is between 800 mAh and 1800 mAh, preferably between 1000 mAh and 1500 mAh, most preferable, approximately or exactly 1100 mAh.

In several embodiments, during the treatment session the fluid inlet of the pump section is in fluid communication with the effluent bag via a tube or line. In other embodiments, the fluid inlet of the pump section is separate from the effluent bag during the treatment session.

In several embodiments, the effluent outlet line includes at least one non-return valve, e.g., upstream of the pump drive. When the effluent bag is disconnected, the non-return valve can advantageously prevent unintentional leakage of effluent from the effluent outlet line, which is beneficial for cleanliness and hygiene.

In several embodiments, the effluent outlet line, which continues downstream of the effluent bag, does not include an element which would be connected to an electrical control device upstream and/or downstream of the pump section of the effluent bag draining apparatus, such as an electrically connected connector. The pump or the pump section are optionally excluded from this; they may be electrically connected. Optionally, however, they are also not electrically connected to, e.g., a control device.

In some embodiments, the pump is configured, for example, as an impeller pump so that it can pump empty an effluent bag filled with ten litres of effluent at at least maximum flow in approximately five minutes or less, preferably, in approximately two minutes.

In several embodiments, the effluent bag draining apparatus according to the present disclosure encompasses a bag holder to hold or receive an effluent bag having an effluent outlet opening. Optionally, the effluent bag or the effluent bag draining apparatus includes a shut-off element for the temporary closing of the effluent outlet opening.

In several embodiments the interruption is made by the control device or closed-loop control device according to the present disclosure, or prompted by the device itself, with the effluent bag still attached, and preferably while it is not yet pumped empty.

Several or all of the embodiments according to the present disclosure, may include one, several, or all of the advantages mentioned above and/or in the following.

An advantage of embodiments of the present disclosure, may be that the draining of the effluent bag can be integrated into the treatment procedure of the patient's extracorporeal blood treatment in such a way that the liquid balancing is not disturbed or falsified during the blood treatment. It is also advantageous not to provide devices which must ensure a continuous and reliable balancing, also during the draining of the effluent bag, with the aim of ensuring an unfalsified balancing.

A further advantage may be that the effluent bag, due to its draining via the effluent bag draining apparatus according to the present disclosure, no longer has to be carried by the hospital staff to a drain, e.g., a washbasin, toilet, plughole, etc. As full effluent bags usually weigh around 10 kg, the hospital staff's workload is considerably reduced if the mobile, separately usable effluent bag draining apparatus is placed, for example, directly next to the blood treatment device and the effluent bag which is to be drained can remain in place on the blood treatment apparatus while it is being drained.

Conventional systems and devices for emptying the effluent bag are sometimes very complex and are also partly subject to approval as medical devices. Some of these systems require, for example, complex dialysis machine hardware, such as special weighing or weighing systems. Retrofitting of such systems with the aim of simplifying the draining of the effluent bag requires changes to the hardware of the blood treatment apparatus, which can be time-consuming and costly for the new approval of the modified machine. For this reason, retrofitting existing blood treatment apparatuses is often not justifiable. Embodiments of the present disclosure make it possible to easily and cost effectively provide a system for draining effluent bags which does not require approval as a medical device and which may also help to avoid the need for renewed approval of the blood treatment apparatus the effluent bag of which should be emptied. Moreover, existing blood treatment apparatuses can be easily supplemented using embodiments of the present disclosure, by positioning the effluent bag draining apparatus according to the present disclosure in the vicinity of the blood treatment apparatus and using it to drain the effluent bag. The control device or closed-loop control device of the blood treatment apparatus must only be programmed for occasional interruptions, e.g., of the balancing, or to transmit information about when such interruptions take place, or when such interruptions are communicated to the effluent bag draining apparatus via signal. Effluent bags known on the market can advantageously be used continuously together with the apparatuses and devices according to the present disclosure.

Embodiments of the present disclosure can advantageously further avoid the risk, when the contents of the effluent bag are drained, of electrical contact between the liquid in the effluent outlet line or another line conveying effluent and the ground, so that the permissible patient leakage currents would be exceeded in the event of a fault, since the effluent bag draining apparatus according to the present disclosure does not itself have to be connected to a power supply. Using the battery-driven pump poses no risk of an electrical accident or voltage damage for the patient.

A further advantage of embodiments of the present disclosure may be that due to the regular to quasi-continuous draining of the effluent bag, previously required bag change intervals for the effluent bag, or intervals for draining the effluent bag, can at least be partially omitted.

A further advantage of embodiments of the present disclosure may be that its use enables work procedures at the blood treatment apparatus to be optimised, since the draining of the full effluent bag can take place, for example, during the already occurring interruptions in treatment.

All of the advantages achievable with the method according to the present disclosure can also be achieved undiminished with the devices according to the present disclosure and vice versa.

### BRIEF DESCRIPTION OF THE FIGURES

The present disclosure is exemplarily explained with regard to the accompanying drawing in which same reference numerals refer to the same or similar components. In the figures of the drawing the following applies:

FIG. 1 shows in simplified representation, a process flow chart of a blood treatment apparatus according to the present disclosure having an extracorporeal blood circuit and a control device or closed-loop control device according to the present disclosure;

FIG. 2 shows an effluent bag draining apparatus according to the present disclosure in a first embodiment; and

FIG. 3 shows a highly simplified diagram of a volume flow in an effluent inlet line of a blood treatment apparatus according to the present disclosure over time (top) and a diagram of the volume flow in the effluent outlet line of an effluent bag draining apparatus according to the present disclosure over time (bottom).

### DETAILED DESCRIPTION

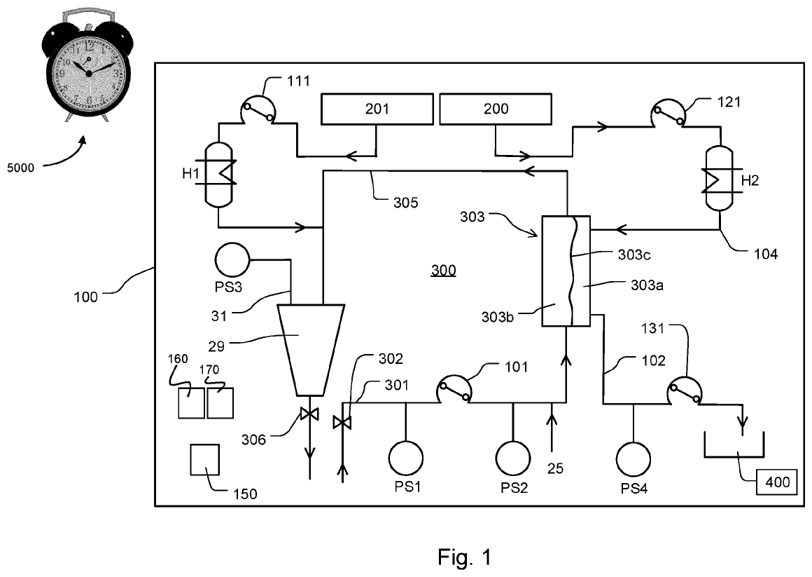

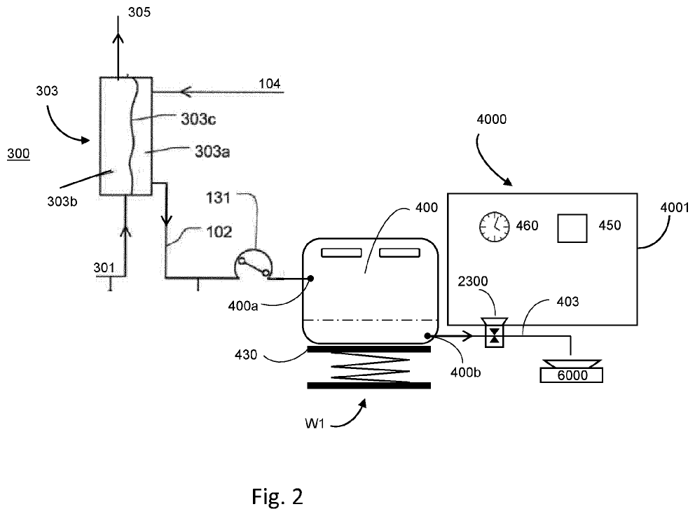

FIG. 1 shows in a highly simplified representation a process flow chart of a blood treatment apparatus 100 according to the present disclosure, optionally connected to an extracorporeal blood circuit 300 and to a drainage tubing system directed to an effluent bag 400. The effluent bag 400 can be part of the blood treatment device 100 or part of the effluent bag draining apparatus 4000 shown in FIG. 2.

The extracorporeal blood circuit 300 includes a first line 301, here in the form of an arterial line section.

The first line 301 is in fluid communication with a blood treatment apparatus, here exemplarily a blood filter or dialyzer 303. The blood filter 303 includes a dialysis liquid chamber 303a and a blood chamber 303b, which are separated from each other by a mostly semi-permeable membrane 303c.

The extracorporeal blood circuit 300 further includes at least a second line 305, here in the form of a venous line section. Both the first line 301 as well as the second line 305 can serve for being connected to the patient's vascular system (not shown).

The first line 301 is optionally connected with a (first) tubing clamp 302 for blocking or closing of line 301. The second line 305 is optionally connected with a (second) tubing clamp 306 for the blocking or closing of line 305.

The blood treatment apparatus 100 represented schematically and only by some of its devices in FIG. 1, includes a blood pump 101. During the patient's treatment the blood pump 101 conveys blood through sections of the extracorporeal blood circuit 300 towards the blood filter or dialyzer 303. This is indicated by the small arrows which are used in each of the figures to generally illustrate the direction of flow.

Using a pump for dialysis liquid 121 that may be embodied as a roller pump or as any otherwise occluding pump, fresh dialysis liquid is pumped from a source 200 along the dialysis liquid inlet line 104 into the dialysis liquid chamber 303a. The dialysis liquid leaves the dialysis liquid chamber 303a as dialysate, possibly enriched with filtrate, towards the effluent bag 400 and will be referred to herein as effluent.

The source 200 may be, for example a bag or a container. The source 200 may also be a fluid line, out of which on-line and/or continuously generated or mixed liquid is provided, for example, a hydraulic outlet or hydraulic port of the blood treatment apparatus 100.

A further source 201 with substituate may be optionally provided. It may correspond to the source 200 or be a separate source.

An only roughly indicated control device or closed-loop control device 150 can be configured to control or regulate the blood treatment session.

The control device or closed-loop control device 150 may be designed to stop a balancing done by the blood treatment apparatus 100 using a clock 160, only roughly indicated, optionally via a signal emitter 170, e.g., at specified times or after specified time intervals.

Alternatively, the clock 160 can be intended to be in signal communication with an external clock 5000, e.g., a network clock, a ward clock or an atomic clock, to be synchronized with such a clock or to be identical to it.

Further, the clock 160 and/or the signal emitter 170 can optionally be provided, as described in FIG. 2, to be in signal communication with a clock of the effluent bag draining apparatus 4000, e.g., to be synchronized with it.

Alternatively or additionally, the signal emitter 170 may be in signal communication with the control device 450 of the effluent bag draining apparatus 4000, in order to prompt, or trigger a draining interval via the pump section 2300, (see FIG. 2).

Where the effluent bag 400 is fluidically, form-fitting and/or force-fitting, optionally connected to the blood treatment apparatus 100 is indicated in the bottom right of FIG. 1. An effluent bag draining apparatus 4000 optionally only fluidically connected to the effluent bag 400 as well as its components are first shown in FIG. 2.

In addition to the aforementioned blood pump 101, the arrangement in FIG. 1 further includes, purely optionally, a series of other pumps, in each case optional, namely the pump 111 for substituate, the pump 121 for dialysis liquid and the pump 131 for the effluent.

The pump 121 is provided to feed dialysis liquid, out of a source 200, for example a bag, via an optional existing bag heater H2 having a heat bag to the blood filter 303, via a dialysis liquid inlet line 104.

The thus supplied dialysis liquid exits the blood filter 303 via a dialysate outlet line 102, supported by the optional pump 131, and may be discarded.

Upstream of blood pump 101 an optional arterial sensor PS1 is provided. It measures the pressure in the arterial line during the patient's treatment.

Downstream of the blood pump 101, but upstream of the blood filter 303 and if provided, upstream of an addition site 25 for Heparin, a further optional pressure sensor PS2 is provided. It measures the pressure upstream of the blood filter 303 (“pre-hemofilter”).

Again, a further pressure sensor may be provided as PS4 downstream of the blood filter 303, however preferably upstream of the pump 131 in the dialysate outlet line 102 to measure the filtrate pressure of the blood filter 303.

Blood, which leaves the blood filter 303, passes through an optional venous blood chamber 29, which can include a ventilation device 31 and/or a further pressure sensor PS3.

The control device or closed-loop control device 150 shown in FIG. 1 can be in cable or wireless signal communication with any of the components referred to herein—at least with the blood pump 101—in order to control or regulate the blood treatment apparatus 100.

The optional pump 111 is provided to feed substituate from the optional source 201, for example a bag, via an optional available bag heater H1 having a heat bag, to the second line 305.

FIG. 2 shows a effluent bag draining apparatus 4000 according to the present disclosure in a first embodiment.

FIG. 2 also shows on the left, a section of the extracorporeal blood circuit 300 of a blood treatment apparatus 100 from FIG. 1, namely the arterial line section 301, which, with reference to FIG. 2, leads from below into the blood chamber 303b of the dialyzer 303. On the opposite side of dialyzer 303 (to the top of FIG. 2), the venous line section 305 leads back out of the blood chamber 303b. Separated from the blood chamber 303b by the semi-permeable membrane 303c is the dialysis liquid chamber 303a, into which fresh dialysis liquid passes via the dialysis liquid inlet line 104 into the dialyzer 303. The dialysis liquid is led downstream of the dialyzer 303, henceforth referred to as filtrate or effluent, using the pump 131 via the dialysate outlet line or effluent inlet line 102 (as it feeds effluent to the effluent bag 400) out from the dialysis liquid chamber 303a towards the effluent bag 400, where it is fed through an effluent inlet opening 400a into an interior of the effluent bag 400. The effluent bag 400 is here exemplarily arranged on or in a bag holder 430, which again is connected to a weighing scale W1 or to another weighing device and may be part of the blood treatment apparatus 100 or effluent bag draining apparatus 4000. Marked with a dot-dash line is an example of a fill level or an example of a liquid level inside the effluent bag 400.

The pump section 2300 is activated by a control device 450 during an interruption interval, during which the balancing by the blood treatment apparatus 100 is stopped and with it also, for example the pump 131. It then sucks the effluent located in the effluent bag 400 or parts thereof through the effluent outlet opening 400b, which is optionally arranged to the bottom right of the effluent bag 400 in FIG. 2, and pumps effluent along an effluent outlet line 403, for example into a basin 6000.

A clock 460 can be provided in the effluent bag draining apparatus 4000 to determine the start of the pumping activity, for instance in order to determine when there is a draining time point. The effluent bag draining apparatus 4000 can optionally be in signal communication with the clock 160 of the blood treatment apparatus 100, the signal emitter 170, or an external clock 5000 (see FIG. 1).

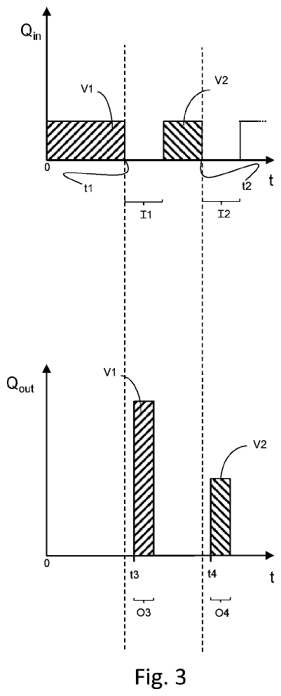

The control device 450 of the effluent bag draining apparatus 4000 may be programmed to start the draining process, at a predetermined time point and exactly for a period of predetermined duration the draining time period, and upon reaching the end of this time period preferably also to end it. If possible, this draining time period preferably will be during, or overlap with an interruption interval in which the blood treatment apparatus 100 is inactive (interruption time period). Preferably, during this draining time period the effluent bag 400 is, in each case, preferably emptied completely (see also FIG. 3).

The term “the blood treatment apparatus 100 is inactive” means in this embodiment, that at this point in time at least one of the pumps 111, 121 and 101 (see FIG. 1) is not conveying and/or the balancing is stopped.

As indicated in FIG. 2, any two or more of the components of the effluent bag draining apparatus 4000, referred to herein, may be in their entirety or partially, optionally arranged in or on a common housing 4001.

Together FIG. 1 and FIG. 2 show a possible embodiment of the system according to the present disclosure.

FIG. 3 shows in its upper diagram, an exemplary volume flow Qᵢₙ over time t generated by the pump 131 in the effluent inlet line 102.

The pump 131 delivers a volume V1 into the effluent bag 400 from the time point t=0 to the time point t=t1, and a volume V2 from the time point t=t1 to the time point t=t2. Hatching is used to display these volumes V1, V2 in the upper diagram of FIG. 2.

It can also be seen from the upper diagram that the pump 131 is stopped at predetermined time points t1 and t2, and in each case is not conveying for the predetermined length or duration of an interval I1 or I2 (interruption intervals are denoted by I as interruption).

I1, I2 are in each case to be understood here as interruption time durations, and t1 and t2 as interruption times points at which I1, I2 each begin.

FIG. 3 shows in its lower diagram a volume flow Qₒᵤₜ with which the pump section 2300 of the effluent outlet line 403 of the effluent bag draining apparatus 4000 delivers effluent out of the effluent bag 400 over the time t.

It can be seen from the lower diagram that the pump section 2300 is activated at predetermined time points t3 and t4 and is conveying for the length of a predetermined interval O3 or O4 (draining intervals are denoted here by O as out).

O3, O4 are here to be understood in each case as draining time periods, and t3 and t4 as draining time points at which O3, O4 each begin.

It can further be seen in the lower diagram that the third time point t3 can be delayed by an optional waiting time after the first time point t1 and the fourth time point t4 can be delayed by an optional waiting period after the second time point t2 (for t1 and t2 see the upper diagram in each case). These time delays can each be of the same length, but they do not have to be. In addition, the intervals O3, O4 are preferably within the intervals I1, I2 or overlap with them.

Preferably, within the intervals O3, O4, the same volumes V1 or V2 are conveyed out of the effluent bag 400 by the pump section 2300, as were previously pumped into the effluent bag 400 by the pump 131 before the intervals I1, I2 (in the upper diagram). These volumes V1, V2 are also shown as hatched areas in the lower diagram.

### LIST OF REFERENCE NUMERALS

25 addition point for Heparin (optional)
29 venous blood chamber (optional)
31 ventilation device (or venting or de-aeration or de-airing device)
100 blood treatment apparatus
101 blood pump
102 dialysate outlet line, effluent inlet line
104 dialysis liquid inlet line
111 pump for substituate
121 pump for dialysis liquid
131 pump for dialysate or effluent in effluent inlet line
150 control device or closed-loop control device
160 clock
170 signal emitter
200 dialysis liquid source
201 substituate source, optional
300 extracorporeal blood circuit
301 first line (arterial line section)
302 (first) tubing clamp
303 blood filter or dialyzer
303a dialysis liquid chamber
303b blood chamber
303c semi-permeable membrane
305 second line (venous line section)
306 (second) tubing clamp
400 effluent bag
400a effluent inlet opening
400b effluent outlet opening
403 effluent outlet line, drain line
430 bag holder
450 control device
460 clock
2300 pump section
4000 effluent bag draining apparatus
4001 housing
5000 clock
6000 sink or basin; drain; outlet;
H1 bag heater having a bag (substituate)
H2 bag heater having a bag (dialysis liquid)
I1, I2 time interval; interruption interval
O3, O4 time interval; draining interval
PS1, PS2 arterial pressure sensor (optional)
PS3 pressure sensor (optional)
PS4 pressure sensor for measuring the filtrate pressure
t1 first time point; interruption time point
t2 second time point; interruption time point
t3 third time point; draining time point
t4 fourth time point; draining time point
V1 first conveying volume
V2 second conveying volume
W1 weighing scales

1-19. (canceled)

20. A control device or closed-loop control device programmed for controlling or regulating a blood treatment apparatus during a treatment session that treats a patient's blood and that is carried out by an extracorporeal blood tubing set, wherein the blood treatment apparatus, while balancing liquid flows, conveying using a filtrate pump, conveying using a substituate pump, and/or conveying, using a dialysis liquid pump, wherein the control device or closed-loop control device is further programmed to, at one or more predetermined interruption time points that fall within the duration of the treatment session:
interrupt a balancing of the liquid flows, and/or
interrupt a calcium solution flow or a conveying action of a calcium pump, a citrate solution flow or a conveying action of a citrate pump, a Heparin solution flow or a conveying action of a Heparin pump, a filtrate flow or a conveying action of the filtrate pump, a substituate flow or a conveying action of a substituate pump, and/or a dialysis fluid flow or a conveying action of a dialysis liquid pump.

21. The control device or closed-loop control device according to claim 20, wherein an interruption takes place for a predetermined interruption period beginning with a corresponding interruption time point.

22. The control device or closed-loop control device according to claim 20, wherein upon reaching the predetermined interruption time point, a signal or alarm may be given to the user prompting the user to manually empty an effluent bag that is in fluid communication with the blood treatment apparatus during the blood treatment session in order to receive effluent that is produced during the treatment session.

23. The control device or closed-loop control device according to claim 20, wherein the control device or closed-loop control device is in signal communication with a signal emitter or with a clock of the control device or closed-loop control device or an external clock of the blood treatment apparatus, a network clock, a ward clock or an atomic clock, and wherein the control device or closed-loop control device is configured, upon receiving a predetermined signal from the signal emitter or upon reaching a predetermined time on the clock, to specify a time that the predetermined signal is received or the predetermined time is reached as an interruption time point.

24. The control device or closed-loop control device according to claim 20, wherein the control device or closed-loop control device is programmed to monitor the effluent bag to ensure that the effluent bag is not filled above a predetermined level or volume.

25. A blood treatment apparatus having a control device or closed-loop control device programmed for controlling or regulating the blood treatment apparatus during a treatment session that treats a patient's blood and that is carried out by an extracorporeal blood tubing set, wherein the blood treatment apparatus, while balancing liquid flows, conveying using a filtrate pump, conveying using a substituate pump, and/or conveying, using a dialysis liquid pump, wherein the control device or closed-loop control device is further programmed to, at one or more predetermined interruption time points that fall within the duration of the treatment session:
interrupt a balancing of the liquid flows, and/or
interrupt a calcium solution flow or a conveying action of a calcium pump, a citrate solution flow or a conveying action of a citrate pump, a Heparin solution flow or a conveying action of a Heparin pump, a filtrate flow or a conveying action of the filtrate pump, a substituate flow or a conveying action of a substituate pump, and/or a dialysis fluid flow or a conveying action of a dialysis liquid pump.

26. The blood treatment apparatus according to claim 25, wherein the blood treatment apparatus comprises a dialysis apparatus, a hemodialysis apparatus, a hemofiltration apparatus or hemodiafiltration apparatus, or an apparatus for chronic renal replacement therapy or for continuous renal replacement therapy.

27. An effluent bag draining apparatus comprising:
an effluent outlet line for guiding effluent out of an effluent bag;
a pump section for conveying effluent out of the effluent bag; and
a control device programmed to prompt the pump section to convey effluent out of the effluent bag when a predetermined draining time point is reached.

28. The effluent bag draining apparatus according to claim 27, wherein the control device is programmed to convey effluent out of the effluent bag via the effluent outlet line using the pump section during a predetermined draining time period.

29. The effluent bag draining apparatus according to claim 27, wherein the control device is in signal communication with a signal emitter or a clock of a blood treatment apparatus, a clock of the effluent bag draining apparatus or an external clock, a network clock, a ward clock, or an atomic clock, and wherein the control device or closed-loop control device is configured, upon receiving a predetermined signal from the signal emitter or upon reaching a predetermined time on the clock, to specify the time at which the predetermined signal is received or the predetermined time reached as the draining time point or to set the predetermined time in relation to this, wherein the blood treatment apparatus comprises a control device or closed-loop control device programmed for controlling or regulating the blood treatment apparatus during a treatment session that treats a patient's blood and that is carried out by an extracorporeal blood tubing set, wherein the blood treatment apparatus, while balancing liquid flows, conveying using a filtrate pump, conveying using a substituate pump, and/or conveying, using a dialysis liquid pump, wherein the control device or closed-loop control device is further programmed to, at one or more predetermined interruption time points that fall within the duration of the treatment session:
interrupt a balancing of the liquid flows, and/or
interrupt a calcium solution flow or a conveying action of a calcium pump, a citrate solution flow or a conveying action of a citrate pump, a Heparin solution flow or a conveying action of a Heparin pump, a filtrate flow or a conveying action of the filtrate pump, a substituate flow or a conveying action of a substituate pump, and/or a dialysis fluid flow or a conveying action of a dialysis liquid pump.

30. A system comprising a blood treatment apparatus and an effluent bag draining apparatus, wherein the blood treatment apparatus comprises a control device or closed-loop control device programmed for controlling or regulating the blood treatment apparatus during a treatment session that treats a patient's blood and that is carried out by an extracorporeal blood tubing set, wherein the blood treatment apparatus, while balancing liquid flows, conveying using a filtrate pump, conveying using a substituate pump, and/or conveying, using a dialysis liquid pump, wherein the control device or closed-loop control device is further programmed to, at one or more predetermined interruption time points that fall within the duration of the treatment session:
interrupt a balancing of the liquid flows, and/or
interrupt a calcium solution flow or a conveying action of a calcium pump, a citrate solution flow or a conveying action of a citrate pump, a Heparin solution flow or a conveying action of a Heparin pump, a filtrate flow or a conveying action of the filtrate pump, a substituate flow or a conveying action of a substituate pump, and/or a dialysis fluid flow or a conveying action of a dialysis liquid pump, and
wherein the effluent bag draining apparatus comprises:
an effluent outlet line for guiding effluent out of an effluent bag;
a pump section for conveying effluent out of the effluent bag; and
a control device programmed to prompt the pump section to convey effluent out of the effluent bag when a predetermined draining time point is reached.

31. The system according to claim 30, wherein the blood treatment apparatus and the effluent bag draining apparatus are separate from each other.

32. The system according to claim 30, wherein the control device or closed-loop control device of the blood treatment apparatus and the control device of the effluent bag draining apparatus are each programmed such that at least one interruption time point and at least one draining time point occur simultaneously or are offset by a predetermined period of time.

33. The system according to claim 30, wherein the control device or closed-loop control device of the blood treatment apparatus and the control device of the effluent bag draining apparatus are each programmed such that at least one interruption time period and at least one draining time period overlap each other by at least 50% of the duration of either interruption time period.

34. The system according to claim 30, wherein the signal emitter or the clock, which is in signal communication with the control device or closed-loop control device of the blood treatment apparatus, and the signal emitter or the clock, which is in signal communication with the control device of the effluent bag draining apparatus, are identical or are synchronised.

35. A method for draining an effluent bag filled with effluent during a patient's blood treatment using a blood treatment apparatus and an effluent bag draining apparatus comprising an effluent outlet line for guiding effluent out of an effluent bag, a pump section for conveying effluent out of the effluent bag, and a control device programmed to prompt the pump section to convey effluent out of the effluent bag when a predetermined draining time point is reached, wherein an effluent bag, which comprises an effluent outlet opening, is in fluid communication with the blood treatment apparatus and/or is connected to the effluent bag draining apparatus, wherein the effluent outlet opening of the effluent bag is connected to the effluent outlet line of the effluent bag draining apparatus to conduct effluent out of the effluent bag, such that a fluid communication is established between an interior space of the effluent bag and an interior space of the effluent outlet line, wherein the method comprises:
at one or more predetermined interruption time points that fall within a duration of a treatment session:
stopping a balancing; and/or
stopping a calcium solution flow or a conveying action of a calcium pump, a citrate solution flow or a conveying action of a citrate pump, a substituate solution flow or a conveying action of a substituate pump, a Heparin solution flow or a conveying action of a Heparin pump, a filtrate flow or a conveying action of a filtrate pump and/or a dialysis liquid flow or a conveying action of the dialysis liquid pump;
and
prompting the pump section to convey effluent out from the effluent bag when a predetermined draining time point is reached.

36. The method according to claim 35, wherein an interruption takes place for a predetermined interruption period.
